(19) United States
(12) Patent Application Publication
(10) Pub. No.: US 2008/0294014 A1
(43) Pub. Date: Nov. 27, 2008

(54) PROCESS FOR DIAGNOSING AND TREATING A PSYCHOLOGICAL CONDITION OR ASSIGNING A PERSONALITY CLASSIFICATION TO AN INDIVIDUAL

(76) Inventor: Barry Goodfield, Glendale, AZ (US)

Correspondence Address: FENNEMORE CRAIG, 3003 NORTH CENTRAL AVENUE, SUITE 2600, PHOENIX, AZ, 85012, US

(21) Appl. No.: 12/124,938

(22) Filed: May 21, 2008

Related U.S. Application Data
(60) Provisional application No. 60/939,183, filed on May 21, 2007.

(51) Int. Cl. A61B 5/00

(52) U.S. Cl. 600/300

(57) ABSTRACT

The present invention is directed to methods of diagnosing a psychological condition and/or the events or perceived events that are the basis of the condition. In another embodiment the present invention is directed to a method of assigning a personality classification to an individual. The methods of the invention preferably comprise the steps of: asking the individual a plurality of questions designed to bring out an emotional response in the individual; observing all repetitive physical movements by the individual while the individual is responding to the questions asked; identifying nonverbal leaks (NVL) from the observed repetitive physical movements reflecting an unresolved perceived trauma in the individual; and diagnosing a psychological condition in the individual or assigning a personality classification to the individual based on the individual's identified NVLs. The present invention is further directed to identifying the underlying cause of the perceived trauma, thereby treating the individual.

### CROSS-REFERENCE TO RELATED PATENT APPLICATIONS

This patent application is based on and claims the benefit of U.S. provisional application Ser. No. 60/939,183, filed May 21, 2007, the content of which is incorporated herein in its entirety by reference thereto.

### FIELD OF THE INVENTION

The present invention is directed to processes of detecting and diagnosing psychological conditions and the underlying causes for the psychological condition in an individual. The present invention is further directed to methods of treating the underlying causes of the psychological condition after diagnosis. Still further the present invention can be used to assign an individual a personality classification.

### BACKGROUND OF THE INVENTION

Many people suffer from undiagnosed psychological conditions caused by unresolved perceived trauma by the individual. It is often difficult to treat these conditions, as the underlying cause is unknown. Furthermore, typically the methods of diagnosis are focused on the conscious awareness of the individual that do not adequately take into account the unconscious expression of past events. Still further, present methods of assessing a person's personality are typically based on the conscious expressions of the individual and very little on the unconscious.

In view of this, a need exist for new and effective methods of diagnosing a psychological condition in an individual and assessing an individual's personality that more fully relies on and takes into account the unconscious expression by the individual. Furthermore, a need exist for a method of identifying the underlying unresolved trauma perceived by the individual that is causing the condition or personality trait so that effective treatment may be rendered to assist the individual in resolving the conflict, thereby treating the psychological condition or improving the individual's personality.

### SUMMARY OF THE INVENTION

The present invention is directed to methods of reading an individual's unconscious expression. In one embodiment, the invention is directed to a method of diagnosing a psychological condition in an individual and/or the events or perceived events that are the basis of the condition. The method preferably comprises the steps of:

placing the individual in direct view of a display device having a real-time frontal image of the individual and/or a recording device for review at a later time;

asking the individual a plurality of questions designed to bring out an emotional response in the individual while the individual watches the real-time image generated on the display and/or while recording the individuals image for review at a later time;

observing and annotating all repetitive physical movements by the individual while the individual is responding to the questions asked;

identifying nonverbal leaks (NVL) from the observed repetitive physical movements reflecting an unresolved perceived trauma in the individual; and

diagnosing a psychological condition or cause of the psychological condition in the individual by correlating the individual's NVL to a psychological condition.

Generally, the display device is a television or computer monitor, wherein the real-time frontal image of the individual is generated by a video recorder or digital camera recording the image of the individual on the display device.

In the most preferred embodiment, the image of the individual is from the shoulders up and is recorded so that it can be viewed at a later time. Preferably, the NVLs are identified by sitting with the individual and watching the recording using slow motion and/or still frame to correlate the repetitive physical movements with the questions asked.

Typically, the most common NVLs that occur during questioning are one of the following: eyes held open, eyes not focused; slack jaw, tongue showing, lowering of head, shallow breathing, shoulders downward, eyes teary, corners of mouth downward, eyes closing, eyebrows up, eyes closed, teary eyes, squinting, tightening of jaw muscles, tightening of upper lip, pressing lips, holding breath, moving tongue in and out, holding mouth open, tightening around the mouth, moving eyebrow(s) up or down, biting down, swallowing, shifting jaw, showing teeth, eyes closing partially, and biting lip or tongue. In addition, each individual typically has a combination of NVLs, which group of NVLs can be further correlated to a condition.

The questions asked during the method are typically designed to invoke emotional feelings in the individual being asked, thereby more likely resulting in the display of the individuals NVLs. Typically, the individual is asked to repeat at least three times their name and the name of their mother, father, or someone important to them, which often lead to the discovery of NVLs.

Advantageously, the present method may be used to identify and diagnosis psychological conditions including mood disorders, anxiety disorders, somatoform disorders, factitious disorders, sleep disorders, eating disorders, impulse/control disorders, adjustment disorders, or personality disorders. More importantly, the present method may be used to further identify the individual's underlying unresolved perceived trauma in the individual leading to a focused treatment to assist the individual in resolving the perceived trauma and thereby treating the underlying cause of the psychological condition.

Further, in another embodiment of the present invention the method can be used to determine and assign a personality type to an individual. Preferably the method comprises the steps of: asking the individual a plurality of questions designed to bring out an emotional response in the individual; observing and annotating all repetitive physical movements by the individual while the individual is responding to the questions asked; identifying from the observed repetitive physical movements nonverbal leaks (NVL) reflecting an unresolved perceived trauma in the individual; and assigning a personality type to the individual by correlating the individual's NVL to a personality type.

Preferably the method utilizes a video recorder for review of the interview session with the individual to further assist in identifying the NVLs. Typically, the personality type is assigned by correlating the NVLs observed by the individual with one of three basic personality distinctions selected from a pre-matrix, a during matrix, and a post matrix grouping. Still further, it is preferable if the personality type is further chosen for those individuals having a personality type assigned to the pre-matrix group by selecting a further sub grouping classification consisting of: the planner, the plotter, the detailer, and the influencer; or for those individuals having a personality type assigned to the during matrix group are further assigned a sub grouping classification consisting of: the doer, the thinker, the pouncer, and the prevailer; or those individuals having a personality type assigned to the post matrix group from a sub grouping classification consisting of: the doubter, the analyst, the inquisitor, and the determinator.

### DETAILED DESCRIPTION OF THE INVENTION

“NVL” (Nonverbal leaks) are defined as unconscious, repetitive, patterned movement from the shoulders up that reflect an old decision or strategy from the past. As taught herein, identification of an individual's NVL can be extremely useful in diagnosing both a psychological condition and the underlying causes thereof.

The method described herein combines basic therapy, hypnosis and video technology to explore the NVL of the individual. Identification and use of NVLs provides far more accurate data than an individual's conscious spoken words can provide. That is, the individual's body is able to say more about him/her than his conscious self is able or willing to express, however honest and complete the individual tries to be. By observing and identifying the NVL signals, the individuals can better understand themselves and the therapist can use the NVLs to diagnose the psychological condition with its underlying causes.

As described above, the present invention is directed to processes of detecting and diagnosing psychological conditions in a subject and further to detecting the underlying causes of the psychological conditions. The present invention is further directed to methods of treating underlying causes of the psychological condition, thereby treating the psychological condition that the individual suffers from.

The method is based on the fact that the NVLs provide observable signals from the unconscious side of the individual. NVLs relate directly to the self-concept of the individual and can be used to determine the individual's psychological strategies, which relate to psychological condition of the individual.

It is assumed in psychology that with stress goes regression. People return to earlier patterns of behavior reflecting the primary strategies they learned to deal with the stress, which comes from unconscious programming. The present invention takes into account both the individual's conscious and unconscious programming.

By asking the individual a series of questions in a systematic way that list emotional responses, the early unconscious programming may be observed by identifying the individual's NVLs. The questions posed, preferably relate to primary relationships and responses to those primary relationships. For example, an individual is typically brought before a display or recording device and asked to say their name. The statement of their name illustrates how they feel about them self's on an unconscious level. Preferably the person is asked the same question at least twice and more preferably at least three times.

If they are denying their own identity or are significantly ambivalent about that identity they will close their eyes immediately after they say their name. This will happen repetitive and is a factor in the initial diagnosis of somebody. If the unusual programming of an individual has taught him both conscious and unconscious that it is inappropriate to express aggressive impulses, the observable NVL will include pulsing of the masseter muscle of the jaw. This typically reflects the desire to control the expressing of the aggression.

Therefore asking a question about which a person might feel aggression or have unresolved aggressive feelings might result in the individual's response being a “pulsing” or repetitive movement in the jaw muscles. This reflects ambivalence and unresolved aggression regarding the person or event being discussed. In the screening session each individual is typically asked not only say their name but also the name of their mother, father and the name of somebody important to them. Each of these questions is preferably asked three times in varied context so that a pattern regarding the early programming can be observed.

The name screening session suggests an evaluation by both parties of each other regarding the appropriateness of developing a clinical relationship. This session can be divided into six general categories.

As used herein, the screening session is basically the first face-to-face meeting between the therapist and the individual. This meeting will cover a variety of areas. Both parties size each other up in terms of first impressions, values, styles, and approaches, etc. The issues that initiate the appointment are discussed. Discussion begins regarding the perceived problem that initiated the contact.

Preferably the conversation then shifts to the unique methodology of the presently disclosed method and the use of display and/or recording device begins. It is important that confidentiality of the individual is established, i.e., tape will be erased when the therapy ends and know body will view the videotapes unless agreed upon by both parties. Next the evaluation process begins. The search for the individual's NVL using video occupies the next half of an hour. Both individual and therapist look at the video to find the individual's Non Verbal Leak. After the individual sees and believes what he sees that he, in fact, has a leak showing from his unconscious, he is ready for the next step. The hypnosis check

To access the inaccessible or unconscious area, contact first needs to be made. The unconscious area of our mind holds the key. In one embodiment of the invention, hypnosis is used for this purpose. Preferably the therapist determines the degree of hypnotizability of the individual by performing a “quick hypnosis” test that indicates the individual's ability to enter a trance like state. The degree to which the altered state can be obtained is in proportion to the individual's ability to surface or divulge the perceived trauma events that are at the core of his psychological condition or maladaptive strategy. If the individual is limited in his ability to alter his state of consciousness, other “tools” such as continued use of the display or image recording device can be further used during treatment.

The screening session winds up with the evaluation and Feedback by the therapist. Both share their reaction to the initial meeting. Here the individual is encouraged to expresses any concerns and asks questions regarding possible treatment. If necessary, important family members may be asked to attend if viewed as helpful and necessary by both therapist and individuals and if it would be helpful in identifying the underlying causes of the psychological condition. Logistical issues regarding how long treatment will last and cost discussed.

As stated above, identifying the NVLs is important in making the diagnosis and treating the individual. As explained above, the NVL is a repetitive, patterned movement preferably from the shoulders up, reflecting an unresolved perceived trauma and creating or reinforcing an old decision or strategy from the past. Identifying the NVL is a way of seeing and understanding the unconscious strategies that the individual presents in his total non-verbal behavior. Understanding why people have these so called “leaks” from their unconscious helps in understanding the important of identifying and using the NVL in treatment. The fact is that much of our interpretations and impressions or programming is formed early in our life experience. It is a kind of programming that is based upon limited information and major generalizations that the individual makes early in life when they have extremely limited experience.

Early events can lead to a fundamental shift in perception and reaction resulting in a new decision about how to see and later react to a similar situation. The fact these powerful programming experiences may take place very early in life (some may occur while still inside the womb); by definition can affect the perceptional field and subsequent actions based upon those distorted impressions and inferences. The unconscious may be thought of as a kind of rudder and keel on the person's ship. The wind may blow affecting the seas around us but the keel and rudder will have a profound effect on the course we take and the success we have in weathering the swells and winds on the surface.

The ability to determine and act upon the powerful messages from deep within the individual has major implications. It brings together in a geometric way the power of the conscious and the unconscious to see, understand and solve that which we only felt before. Many questions previously obscured by the unconscious become clear when the present method of analysis and evaluation is employed.

The NVL provides a window into the unconscious communication given off by the individual. When a person becomes able to see these organized unconscious messages he becomes able to clarify that which heretofore was denied and communicate with the unconscious. When the non-verbal leak of an individual is accurately deduced his underlying motivation in most all situations becomes apparent and can be treated.

To establish the leak from a person's unconscious, the individual is asked a series of seemingly unimportant questions—i.e., what is your name, the name of someone important to you and how did you call your mother and father when you where a child, etc. The information gathered here when repeated a few times revels the emotions we have about these important figures in our life through NVLs. What is more important is the fact that it also shows our deeper feelings by the often double messages we exhibit while saying these names. If there is some ambivalence in our feelings towards those individuals and we are reluctant to acknowledge this ambivalence it will likely be shown in our non-verbal behavior. The ambivalence has an unconscious component, as the precipitating factor at the heart of the event(s) is likely below the person's awareness. The individual strives for balance by expressing all feelings, including those that are repressed, some consciously and others unconsciously by means of NVLs. For example, ambivalent or double messages come out as a series of repetitive movements of which we are not consciously aware. The expression of these movements helps our system to establish a homeostasis and hence, create a balance in our total communication process.

In a preferred embodiment, the therapist sits with the individual and watches the recording of the individual during questioning. In one embodiment, the slow motion and still frame are used to discuss with the individual the repetitive movements and to more accurately find the NVLs. The NVLs reflect the unresolved perceived trauma of the individual and typically create and/or reinforce an old decision or strategy for dealing with the stressful situations from the past.

The purpose of asking a series of personal questions is to bring out an emotional response in the individual. The fundamental notion is that traumas are learning points for people, as are non-traumatic events. The emphasis is on trauma because decisions regarding causality and consequence often follow traumas and profoundly impact sequent behavior. In fact, when decisions are made after traumatic events—especially unconscious decisions—they become personality cornerstones on an unconscious level. One's perception of traumatic events drastically influences decisions regarding such basic factors as risk taking, openness to difference, expression of aggression, and contact with others (including love, sexuality, and most emotions). These events are called perceived traumatic events (PTEs). At an unconscious level, one's PTEs form the basis of his or her unconscious self-concept. When an individual's unconscious PTEs is determined by observation of the NVLs, the key to the individual's unconscious process and their personality type is in hand, allowing for the accurate diagnosis of the individual's psychological condition and/or personality type.

Another helpful tool is to make contracts with the individual after the NVLs are identified. A contract is a tool that relates to a mutually understood and agreed upon series of behaviors. It is either general or specific. One of the main factors that lead people into therapy are the lack of clear agreements and a failure to keep the agreements that are made.

Clarity, honesty and respect are critical factors in a relationship and psychotherapy in particular. The contract will provide a general feeling of security between the therapist and individuals. The roles within the therapeutic process are made clear and boundaries are set. The safer the individual feels the greater is the possibility that areas avoided or previously unexplored will get the attention necessary. Risk taking behavior is in direct proportion to growth and clear agreements and clear role expectation certainly provides the necessary base for those actions.

Therapy is an encounter the person has with himself or herself. It is an encounter the individual has with the SL1, SL2, or SL3. This means that a clinical plan selecting either one of the three Symbolic Strategies as the next most logical step.

The decision and the direction comes as a result of a deep conversation bases upon prior sessions and what seem like the logical next step to decode the unconscious process. Once the decision is made a contract is set for this session. The Impact or SL-1 working session would try to find the organ of the trauma or PORT issue (Primary Organ of the Trauma). The Primary Emotion Session or SL-2 is designed to surface the emotion that the individual wanted to express at the time of the PTE. The Primary Coping Strategy or SL-3 Session revolves around how the individual had come to grips with the feeling wanting to be expressed in his SL-2 and how he ultimately decided to express these emotions. So with SL-1, SL-2 and SL-3 established in the initial screening session and refined in a subsequent session. The three areas of the person that are most associated with the traumas are the intrapsychic (how your thought process works); the psycho physiological (how your body reacts on a physical level to the PTE, and interpersonal (how the PTE relates to your relationship with others).

For a working contract to be correct it must incorporate SL-1, SL-2 and SL-3 and the three possible levels of involvement, intra psychic, psycho physiological and interpersonal. Once these variables are determined and organized and understood clearly by the individual a contract is made with the individual.

The last part of this working contract is the identifying the part of the individual's body that he or she feels is most involved in the aspect of the work that is about to begin. The contract to work and the focus of the work for that session makes for clarity and therefore, reduces anxiety, which of course is natural in therapy. Basic to the idea of a contract is the notion of change in thought and action. For people in “crisis” change is in a certain way unnaturally. The contract helps the individual to take a risk, reevaluating the basic strategies. The therapist regularly makes agreements or contracts as it is referred to in the work. There is a general contract (psychological umbrella) that relates to behavior, expectations, methods, unique techniques costs, limits on the part of all parties. As people approach the real idea of change in a therapeutic setting there is a natural anxiety regarding what will happen. Will this “work.” The unconscious is saying, “here we go again another false start, this will not work and do you really want it to?”

The more the structure of the therapeutic process can fight the normal contraction reaction at the beginning of the process the greater is the chance of success occurring. That is why during the screening session the guidelines associated with the present method are clearly explained. Some of those issues discussed and agreed upon prior to therapy beginning are the following:
The therapist has the right to use the video, the individual
Precision bodywork is an important aspect of decoding
Perceived Traumatic Events
Video is used for diagnosis and identification of the NVL
Video is also used treatment in psychotherapy
A clear contract will proceed all clinical working sessions

Preferably the contract is general and specific. The general aspects setting forth the ground rules by which all session will be conducted, i.e., ethics, confidentiality, etc. and the specific aspects setting out as clearly as possible the content to be explored during the sessions. The contract is preferably focused upon the role of SL-1, SL-2 SL-3 and on the intra psychic issues, psychophysicological issues and/or interpersonal issues.

Use of hypnosis is important to facilitate the connection and communication with the unconscious. Altered states of consciousness, or hypnosis, are nothing more than an altered state of awareness. When an individual is in an altered state of conscience he is able to access more of his system. To the extent that our unconscious process is by definition below our level of awareness, it is important to gain entry to where the problem is stored. The events that are traumatizing such as loss of a loved one or tragic accidents or anything that we perceived as traumatic can exist in our memory on all levels. It is important to remember that these memories are perceptions of the events and not the accurate representations of the actual dynamics of the event. They are perceptions not reality. For the person, however, it is “as if” it is reality.

Preferably, when hypnosis is used with the method, each session is systematically planned to interlink with the previous one to form a seamless logical program to decode the PTEs that brought the individual to therapy. There are a variety of induction methods for hypnosis. The standard methods of clinical hypnosis are that which is classically used when an individual goes into the altered state. One preferred method of inducing hypnosis is to ask the individual to sit directly in front of the video screen with a video camera positioned directly behind the monitor. The therapist places their hand on the focus ring and manipulates it in and out of focus. The individual is instructed to try to keep the image in focus in his mind. He is further instructed that when he does this his eyes will become heavier and at a certain point it will be just easier to close his eyes. When he does this the prerecorded material is still framed and the individual is instructed to open his eyes where he will be confronted with a picture of a person with whom is associated with his PTE. This could be a person who he was never able to reach closure prior to his death. It could be a parent or loved one whom he never had the opportunity to say goodbye. This is a very powerful technique and almost always results in a very cathartic reaction and long lasting results as the PTE is directly confronted. A variation on this method can be used with rape victims, victims of physical violence, but should be used careful and with full disclosure and rules between the therapist and the individual.

Begin using the most desirable induction method. Depending upon what has been discussed and agreed upon in the proceeding clinical planning phase, the most appropriate method of induction is chosen. If for example the individual has major trust issues, an induction that will mechanically help to alter his state of consciousness is selected. The individual may be asked to elevate his eyes up into his head and hold them there while slowly closing his eyelids. This method of induction very quickly alters his metabolic process and eases him naturally into an altered state of consciousness. He then will be asked to select a “yes finger” as well as a finger to indicate that he is going deeper into an altered state. The individual is in a certain way on “automatic” and will be less inclined to fight the altered state as he is in charge and determining his own level and pace.

Using the data from the specific contract and the most appropriate method of the altered state of awareness the work begins. The therapeutic session is now focused. The method is clear and the individual is ready to do what has been discussed. Of course, there will be nuances and unique factors within each session as all people differ. What will not differ is the agreement and approach. As the individual enters the altered state of consciousness the therapist is closely watching the subtle signs indicated in the individual's body movement. These subtle movements can be a kind of predictor of what will be coming. They may also indicate how the individual is reacting to the induction method. The goal is to achieve an altered state and close observation and appropriate modification will make this goal a reality.

It is important for the individual to be able to hear the therapist as he is the guide in the session. It is therefore, critical that the individual hear clearly the directives of the therapist. Sometimes the therapist get so involved in the process of what the individual is doing that he makes his voice soft like he is whispering to the unconscious instead or the total individual himself.

The individual is asked to recall the still frame that has been selected by both the individual and himself that represent on a symbolic level the impact, primary emotion or the primary coping strategy. Once this has been done the individual is asked to recall the time and place associated with the PTE. It is assumed that finding the specific time and place or getting to a representative time and place will lead the individual to deduce the basic decision that he took then.

Once the altered state is reached the therapist moves to the next step, which preferably is the use of specific body work to explore the PTEs. The contract is the guide to what happens at this point of the work. Here the therapist skillfully using minimal physical contact with the individual helps the individual to reach his clinical destination. In most cases little pressure is necessary. The therapist maintains excellent position of his own body that has two effects on the therapy process. He sees more of what is happening by positing his body midway between the individual's body. By positing himself properly he also is maintaining a clear awareness of the overall process. This awareness is referred to as the “Meta Level.”

Accuracy in touching the individual makes for a clearer picture of what the individual perceived as the trauma that affected his life. This may be something like the weight of a pencil on a table. Counter pressure is the key to successful bodywork. The therapist never touches the individual with a random touch. The contact is professional and precise.

As the individual reaches the moment where trauma turned truth to a twisted reality a new maladaptive decision is made. At first the decision, in the individual's view, is logical and correct. He sees his action as appropriate in relationship to the situation in which he perceives himself. The most important moment in the attempt find the individual's old decision in the altered state is the moment referred to as the “breathing drop.” The Breathing Drop is the moment where the individual confronts the necessity to take a decision. That moment bring together a number of both psychological and psychophysical reactions within the individual.

The clearest way to see this is to observe the respiratory system. A “BD”, i.e., a breathing drop, reflects the moment just before the decision is taken. It also shows the perceived thought process of the individual at that moment. It is the moment just before the heretofore-repressed emotions are given expression within the session. The individual is most clear in his thought process at that moment.

After seeing the BD the therapist attempts to exact the decision in the most precise way possible. “What did you say to yourself at that time?” “Say your old decision.” The individual begins to say what he thought at the moment of the perceived trauma. Emotions flood out as the impact hits the individual on both a conscious and unconscious level. It is here where precision and ethical bodywork reduces old historical pressures within the individual's system.

The Old Decision is important as it forms the basis for a change in perception of the individual. It is not so much the decision as it is the long lasting implications of that decision. The resultant strategy becomes the problem. The PTE becomes the base for a new way of relating to his environment.

It is essential for the individual to become clear about the old strategy that he took as a result of the PTE. The awareness “frees” him in a way from the guilt of his previous actions as they are now understood in a new context. All of the pain, misgivings and confusion are preferably explored in the session while in the altered state and later in front of the video so all unresolved thoughts and feeling are discussed and re-symbolized in a new and more adaptive manor in relation to his life in the here and now.

The New Decision is a major step in the restructuring of the individual's unconscious self-concept. It is therefore, critical that the New Decision aspect of the therapeutic process be clearly understood. The most difficult learning to overcome is old learning. Therefore, a major revaluation of how to look and act in various situations must be clear to the individual. The New Decision implies a new perspective on means and methods of relating to the new world that is opening up to him. There may be some reluctance to go over the PTE as it is history and bring it up again can on some level be painful of uncomfortable. Regardless of this, it is necessary to rehash the event to reinforce the fact that that was then and this is now. The individual may surface new perspectives that are important to discuss. For example, the implications that the new decision may have for him in his work or relationship with his family. The clearer the individual is about the implications and consequences of his new decision the less he is likely to regress to earlier ways of behaving. He is also more likely to be aware of his actions at the moment when old strategies used to misdirect his actions.

Preferably, a new decision strategy is established that is more appropriate than the old. The new decision is the first step to a less dominant unconscious self-concept. Just as the New Decision is a major step in the clinical process the New Strategy can be thought of as one of the final steps in the clinical process. It may not be the last step as the unconscious self-concept is a conglomerate of PTEs that form the picture that the individual has of himself.

The New Decision is the new approach that will be taken to action whereas the New Strategy is the new practical plan of action. It is important that this distinction be clearly understood. It is the difference between insight and action.

It is very important that the New Strategy be completely understood regarding the implications and consequences. These “wheels of action” turn the individual's life in a new more adaptive direction. He sees and hears more of the things that heretofore he denied to awareness as a result of the blocking effect of the old decision.

The New Strategy leads him to look at other logical inconsistencies in his approach to life. He may realize that acting upon his new decision and strategy may mean that he may have to rethink other areas and actions that he never considered changing before. For example if the new decision is to be more open to change and the new strategy was to say more clearly what he feels he may be forced to be more open to divergent feedback that he in the past normally blocked out as a result of his old self concept.

This systematic decoding of the PTE'S and the individuals self-concept is what is called clinical planning. That is the next step if there is a next step in the individual's clinical treatment.

The present invention will now be illustrated by the following non-limiting examples.

### EXAMPLES

The present invention is directed to methods of diagnosing a psychological condition in an individual and the events or perceived events that are the basis of the condition. It is possible to generalize these basic emotional strategies to personal and professional levels. When collectively brought together and organized it is possible to establish a pattern, which can be associated with 12 basic personality types.

In one embodiment, the NVLs are used to determine a person's personality formation as it relates to their perception of a perceived traumatic event. A personality matrix may be used in this embodiment. The personality matrix may be thought of as three general grouping of four distinct personality types. The groupings are designed to help the individual attempting to do personality assessment with an easy means of capturing general trends and unique differences in the individual's personality. The three general groupings are 1) the Pre-matrix, 2) the During Matrix, and 3) the Post Matrix. Preferably each general group is further divided in to four subgroups each for simplicity purposes. In a preferred embodiment, the groupings are:

### The Pre-Matrix (1)

1.1 The Type One Personality—The Planner

1.4 The Type Four Personality—The Plotter

1.5 The Type Five Personality—The Detailer

1.10 The Type Ten Personality—The Influencer

### The During Matrix (2)

2.2 The Type Two Personality—The Doer

2.6 The Type Six Personality—The Thinker

2.7 The Type Seven Personality—The Pouncer

2.11 The Type Eleven Personality—The Pervailer

### The Post Matrix (3)

3.3 The Type Three Personality—The Doubter

3.8 The Type Eight Personality—The Analysist

3.9 The Type Nine Personality—The Inquisitor

3.12 The Type Twelve Personality—The Determinator

The fundamental core of personality is also a matter of perception and childhood experiences, that is, the early programming experiences (the birth process, etc.) that will determine how life's issues are seen and how they are ultimately solved. If the perception of the early database can be determined via the identification of the individuals NVLs it becomes possible to infer the basic response strategy of the individual to major events experienced.

The aspect of this early programming is most clearly seen and therefore adjusted when deemed necessary by precise observation of the non-verbal queue or “leaks” shown by the individual. These NVLs reflect the core aspects of the early programming strategies and therefore, form the base of the person's personality formation as it relates to their perception of a perceived traumatic event. There is ultimately a reality base to most everything at the same time it is also clear that perception of reality is just that perception. To the extent that this is true it can be said that context determines meaning and perception determines reality. The NVL reflects the unconscious message of the individual as well as the influence of one of the personality types within one of the three matrixes. Each one of the three matrixes has four distinct sub categories, which reflect how each unique personality type relates to the world. With each personality type there are three additional sub categories. These distinctions relate to the symbolic level of awareness. They are Impact, Primary Emotion and Primary Coping Strategy

The Impact (SL-1) is rooted in any perceived traumatic event (PTE). It is real in the eyes of the person who experienced it. This shock to the system can be recorded on both levels of consciousness and unconscious. Moreover, it impacts upon the person on intra-psychic, psychophysiological or interpersonal levels.

The Primary Emotion (SL-2) to the Impact is the first impulse that the person has to the traumatic event. It is what he really wants to do, but not what he or she actually does. If his response to the situation is anger in the SL-2 stage, he wants to express it clearly and directly with no holding back. The Primary Coping Strategy (SL-3) is what the person really does. It is not what he wants to do. He may feel rage but he realizes that, if he gives in to these feelings, he could make the situation even worse or if he feels sadness the expression of those feelings could cause the situation to exacerbate somehow.

This is where the person finds a compromise that works for him at that moment. In psychoanalytic theory the concept of the id, ego and superego seek to explain the decision making process. Freud's ideas are hypothetical constructs that add understanding to the decision making process. Impact, Primary Emotion and Primary Coping Strategy also help to decode the decision making process; however the SL-1 SL-2 and SL-3 are observable, testable realities. With time and similar perceptions of what the world is like, a person develops repetitive strategies for similar situations. It could be said that the SL-3 is a person's basic strategy in dealing with those feeling in life; moreover its roots can be traced back to unconscious observable behavior.

The primary database of the person is the process of birth itself. During the first few years of life that information base is critical. Of course, mother and father are also imperative in their presents or conversely their absence. What however, it also important is the first awareness of life itself the infant. It can be thought of as a filter where sunglasses are necessary for us on a bright day. We ware them and avoid the harsh intrusion of light that is too bright. It does not block the events it simply makes seeing easier and more acceptable.

The body can be thought of as a government with different divisions working in harmony to produce balance and tranquility in all its systems. The conscious system of the body is primarily involved in daily functions. The unconscious is focused more on past perceived traumatic events, and trying to protect the system from suffering from them again.

The three primary matrixes provide the filtering necessary that helps to clarify events and the motivations behind those events. It is important to keep in mind that the initial perceptions formed at birth will determine the filters developed. The realities deduced by the individual are in fact the primary basic unconscious management system that guides human behavior and has forged our history on this planet.

When we look at the non-verbal leak, it is a system between systems designed to maintain homeostasis, or balance, within the body and the individual subsystems of which it is composed. Traumatic and non-traumatic events in the child's life are filtered through the initial program developed in the pre, during, and post birth phases of life.

The three basic experiences (pre, during, post) and the interactive combinations of those experiences lead to twelve unique factors or filters in the formation of personality. It is through these filters that the person abstracts, via perception, the world in which life proceeds.

Both the biological and psychological systems of the body have as a common goal the reinforcement, maintenance, enhancement, and safety of the person. The biological system must and does, co-exist with the psychological system.

Language is the individual's means to develop, maintain, and enhance the conscious and unconscious self-concepts, but those efforts are sometimes working at cross-purposes. For example, an individual may tell someone he is not angry using words that reflect his conscious self-concept, while on an unconscious level, he may send a contrary message, which reflects a perceived traumatic event (PTE) that finds its origin in the past. This will result in an observable Non-Verbal Leak, which is a statement of the true unconscious emotion.

The fact that this NVL is observable provides a practical tool for those involved in separating fact from conscious fiction. The unconscious can be seen and when it is via the NVL deception and disingenuous statements become as easy to read as it is to say Lancelot's remark from Shakespeare's, “The merchant of Venice, “Truth will out.”

Both conscious and unconscious self-concepts reflect a persons attempt to gratify basic needs. These basic needs are all filtered by perception. We all need love, contact, safety, etc and all of these needs are obtained via our perception of reality on both conscious and unconscious levels.

Conversely, those factors, which we feel are unfair or painful, are also recorded within our systems by way of our perceptual fields. A unique NVL program makes defense mechanisms come into play when events block our basic needs or those of significant others. Each of the twelve personality types are unique and warrant an individual explanation.

Not only does the NVL show the unconscious in the upper part of the body, it is also shown with the total body. The unconscious when seen in the total body movement is called the Body Non-Verbal Leak or BNVL. It too is observable and discernable as a unique statement of the unconscious process.

### Matrix One Personality Type 1.1 “The Planner”

If you want a reliable, trustworthy, honest person this is the Type 1.1

The Type One or “Planner” Type Personality is someone who carefully plans and evaluates the development of an area or potential action. He makes his plan and sticks with it regardless of the pressure to change or modify his course.

This is a person who maintains a low profile in relationship to the other personality types; but he is capable of great loyalty regarding family and friends. He does not have high expectations regarding achievement and therefore is non-confrontational regarding his friends, colleagues and co-workers.

He is pleasant and non-threatening to others. He is often seen as a quiet and reliable person whom is easy to be around. He may have a deeper understanding of issues and events than other people may have but he will be somewhat reluctant to impose his observations on others regardless of their validity. The “Planner” is not a complainer; he may accept the unacceptable to avoid conflict or confrontation

Perhaps the most important issue about this person is the danger of overlooking his presence and therefore, missing his input and observations. He will not impose himself, in fact he is seen by many as shy, he must be encouraged to state his ideas and feeling lest he remain quite and his well thought out observations be missed.

Statement Of The NVL On The Symbolic Level For Personality Type 1.1 “I am in pain, feel angry, cover it with sadness, but I hold it in.”

### Matrix One Personality Type 1.4 “The Plotter”

In general the Restrained/Responsive or Type Four or “Plotter Personality” is capable of making systematic steps to obtain his goals or objectives. His determination and willpower make him a person who can achieve his goals, perhaps not as fast as some may achieve theirs, but one would be foolish to assume a lack of speed means that he will be stopped or swayed from his objectives. He will obtain them regardless of price, position, or problems.

His dependability is his strong attribute. When he chooses to use his potential power, you have a person who is capable of turning long-term objectives into tangible, long range, achievable goals.

He is a person who makes secret plans, especially toward activities that may be subversive or devious in some ways. This is related to his prohibition about confronting others when they overstep his boundaries. He will muster his resources when he feels his views and values are under attack or might be; and he will make deep commitments to defend them.

Although focus is a regular issue in “the Plotter” or Restrained/responsive personality, he when focused, can and will deliver.

Statement Of The NVL On The Symbolic Level For Personality Type 1.4 “I am denying myself, that makes me feel sad and also angry, I do not trust myself expressing my feelings and that makes me unsure.”

### Matrix One Personality Type 1.5 “The Detailer”

The “Detailer” Personality is focused on all or many of the particular elements of something or someone. Nothing slips by this person, but he may be reluctant to bring them up himself.

The subtle nuances of a person's expression or an inconstant remark do not slip by this keen observer of life. Although somewhat withdrawn or shy in his presentation, he is capable of holding responsible positions.

He works best when assigned to analyzing tasks or projects on his own. He tends to work best by himself as his contacts with others can be complicated by his desire to please or be seen as capable or up to the task set before him.

When working in conjunction with a Type 1.5 or “Detailer” Personality the colleague would do well to listen to the remarks expressed by him. His lack of verbosity is in no way an accurate reflection or his lack of observational skill or clear and often strong position held on a person, issue or event. The expression, “still waters run deep” is an accurate representation of the personality traits of this quiet and complex person. In the right position this person can be an extremely valuable employee. Others often best present his valuable observations as he can and often is misunderstood by those who do not know him well.

Statement Of The NVL On The Symbolic Level For Personality Type 1.5 “I am in pain that makes me angry I don't feel authorized to express it so I swallow it down and control it.”

### Matrix One Personality Type 1.10 “The Influencer”

“The Influencer” is a charming, strong and disarming personality and very easy to misunderstand. What he lacks in direct messages regarding fulfillment of needs he circumvents by enlisting the support of others to help him in the realization of goals and objectives. In that sense they have the capacity to influence others actions in a very powerful way.

The Scarlet O'Hara character in “Gone with the wind” is an excellent example of powerful powerlessness. It is an example of the use of a “no power” approach to using the power of others. This person is disarming in that he could be seen as powerless but a close look at what happens.

There is another aspect of the “The Influencer” Matrix 1.10 and that is the capacity to plan and wait for the “right” time to take action. This person is also like the frog sitting on a lily pad, waiting, unmoving and calm and than suddenly a tongue comes shooting out and the fly a long distance away disappears.

Between his ability enlist others in his objectives and his ability to plan and strike at the correct moment he is skilled and should not be understated, as he is an extremely strong but disarming personality. This person is charming and influential in a way that denies both. It's seduction by powerlessness—it's a lovely velvet glove covering an iron fist.

Statement Of The NVL On The Symbolic Level For Personality Type 1.10

“I am sad and feel powerless to change this so I wait until I feel the time is right to get my needs filled and then I act.”

### The During Matrix

Matrix Two Personality Type 2.2 “The Doer”

In general, the Type Two “Doer” Personality or is someone who is active in doing things rather than just thinking or talking about him or her. He is a dynamo influencing those around him to engage in actions that they might never embark upon without his

Sense of direction, drive and purpose. “The Doer” is a natural leader who inspires others to action now. He is powerful and often fast to act, sometimes too fast for his own good. When things do go wrong, he is quick to re-evaluate and adopt new strategies to obtain his goals. His charismatic style is infectious, inspiring others with lesser sense of assuredness to follow his leadership. He is well focused and capable of great concentration and inspiring it in others.

The Type 2.2 or Doer Personality radiates strength even when it is not warranted. He, therefore, surprises others when his programs or projects are less than successful. He is capable of great focus and dedication to his tasks and this attribute can be ultimately detrimental to his lifestyle.

Statement Of The NVL On The Symbolic Level For Personality Type 2.2 “I am shocked and in fear, I am sad and cover it with anger, I swallow it down and distance myself.”

### Matrix Two Personality Type 2.6 “The Thinker”

The Type 2.6 or “Thinker” personality is capable of using his mind to reason or reflect and to form thoughts about feelings. This skill is so powerful that it often leads others to see him as a powerful and very “feeling” person. His thoughts about feelings make him a mighty force to be reckoned with when logic is the issue. This person has the capacity to inspire others with his blend of logic and feelings. His reluctance to jump into situations may be seen by others as the use of prudence in the face a need for action. He may be found in the role of a mediator as a result of his reluctance to take one side over another. He has the capacity to see both sides of an issue and there for might be sought out as a negotiator by disputing factions in an argument.

The Type Six or “Thinker” personality is sought out for advice and judgment in difficult situations as he is seen as a person who is capable of having a meta level awareness. He is a reluctant leader, which makes him often the choice for a leadership position when other more assertive people have attempted to resolve a situation using force or arbitrary means of conflict resolution.

He may be seen as an unfocused leader with power, however this strategy has its advantages in some situations. Others may perceive it as prudent, reflective and contemplative in the face of pressure to take action.

Statement Of The NVL On The Symbolic Level For Personality Type 2.2 “I have pain and I am angry, I cannot show it, so I keep it inside and I swallow it down.”

### Matrix Two Personality Type 2.7 “The Pouncer”

The Type Seven or “Pouncer” Personality is a “witness” who does not taking part in the events that go on around him. This ability to stand back and observe individuals and events makes him a powerful person when it comes to facts, information and data about people and places. He is often a critical player in major decision-making situations. He is often found at the center of policy decisions. His strong ego strength often puts him in the center of the fray when action is required. The “Type Seven or “Pouncer” Personality knows what is necessary to obtain his objectives but, many see him watching others actions rather than leading the group. He is a keen observer and insightful about the dynamics of the situations in which he becomes involved. When he feels the time is “right,” he will not hesitate to speak up and notice quickly who agrees with his viewpoint and who does not. When he wants to be, he can be the center of attention showing his power, skills and outstanding organizational abilities. He often privately admits that he feels as though he is living his life behind a glass wall able to see but limited in his ability to be a full participant.

Statement Of The NVL On The Symbolic Level For Personality Type 2.7 “I am shocked and want to express my anger, instead I hold it inside until I can find the correct opportunity to express it fully.”

### Matrix Two Personality Type 2.11 “The Prevailer”

The Prevailer Personality is a natural sales person. This master at the art of influence has skill and power to influence those around himself. Subtle charm, and persuasion are characteristics of this person. The Matrix 2.11 person fissile with words and concepts and is a natural when it comes to communicating them to others.

He is most clearly focused most on the gratification of his needs and his goals and objectives. When they are consistent with corporate objectives he is a dynamo.

The type 11 is often successful in work situations as a result of his keen ability to perceive the needs of others.

Action is more of a priority than intimacy he is therefore, often unsuccessful in gratifying his needs in interpersonal relationship requiring intimacy. This is due to the fundamental distrust he has regarding contact. He may have either multiple relationships of avoid them all together and put his efforts in his work. The Prevailer Personality is a theoretical expert on people and their needs.

He does however; suffer from a lack of contact and emotional gratification of his basic needs. The good news is that he is an expert in facilitating the needs of others and he clearly sees that and his inability to achieve that in his own life is often the bad news. He has in that sense, something in common with the Type Seven or “Pouncer” Personality (2.7).

He is watching the world he helps to create go by in front of his eyes. This charming pleasant leader is alone and often lonely, a fact he skillfully hides from others as his life goes by.

Statement Of The NVL On The Symbolic Level For Personality Type 2.11 “I am in alone and in pain, that makes me angry, but I distrust those feelings so I swallow them down put on a fake smile and seduce those around me.”

### Post Matrix (Three)

### The Type Three Personality

### Matrix Three Personality Type 3.3 “The Doubter”

He has a large sense of duty if he is involved with persons or things He is extremely loyal with people that he trusts. If someone criticizes him he feels attacked, which makes him insecure He likes it to belong to a group, it gives a secure feeling he is a mismatched. He is strongly directed to everything that can mean danger and has also a certain fear for success. Part of his fear emerges from his rich imagination about what can happen and go wrong.

His self-concept is to do his duty, display good behavior He can be seen as sympathetic and to meet approval When he is afraid: to be comforted, to reinstall his certitude and to let the person with authority help him He prefers to be alone instead of lonely. His favorite style of leadership is the ‘team player’ He prevails the well being of his team before his own interests

Statement Of The NVL On The Symbolic Level For Personality Type 3.3 “I'm in pain; I don't belief what happened to me, I am angry. I'm not authorized to express it so I control it by holding in.”

### Matrix Three Personality Type 3.8 “The Analyst”

He is a very trustworthy person as he understands the rules of the game and will follow a leader who he feels has the power to carry out that which he himself sees as appropriate but unable to do on his own. As a leader, his basic distrust of his own power makes him a reluctant leader who distrusts his ability to lead. His non-verbal behavior could show mild trance.

Her mind is always working. She shares about 1/10th of what she feels but to a very selected few she is an open book. Her thinking and logic is outstanding and very clear. She prides herself as being fair and open to difference. Although she holds herself to a very high standard she understands the difficulties associated in achieving and maintaining those standards.

This person takes life seriously, is responsible and compassionate. Few games go by her unnoticed or unpunished when deemed appropriate.

Statement Of The NVL On The Symbolic Level For Personality Type 3.8 “I feel Pain and Disbelief, it makes me sad and angry, I do not want to express it so I wait and watch what you do and then express the anger and swallow the sad feelings down.”

### Matrix Three Personality Type 3.9 “The Inquisitor”

The Type 3.9 or INQUISITOR Personality is intense, focused and distrusting. He is a person who asks a succession of relentless and searching, often perceived as hostile, questions to determine what for him is the “truth.”

Tough, strong and determined, he sees himself and is often seen as a “winner.” He has real power to lead, influence and persuade others to endorse his positions or viewpoints in general. This person migrates toward power and is very comfortable in leadership positions.

The Inquisitor Personality is also a trustworthy individual who often gathers numerous individuals around him who look to him for leadership or guidance. This person is the quintessential leader, self-assured, calm in crises, revealing controversy with a sense of direction that assumes that his perception of the facts are the undeniable “truth” itself. He has a history of risk taking and often a series of successes that validate the decisions behind the risks taken. Add intelligence and education to “the Inquisitor” and “the sky's the limit.”

Statement Of The NVL On The Symbolic Level For Personality Type 3.9 “I feel pain and fear, and become angry, my question is shall I show this . . . or distance myself.”

### Matrix Three Personality Type 3.12 “The Determinator”

Bright, powerful, with a strong but rigid ego if order is the game “the Determinator” is the name. The Matrix 3.12 can become frustrated when events and people fail to live up to expectations. He loves perfection, strives to find it, and never does; therefore, he is often frustrated.

This is a hard working individual seeks symmetry, fighting for balance, and suffering the fact that it is generally illusive. This person is reluctant to confide deep feelings that he harbors with other as a result of old disappointments and past failures regarding intimacy.

Often found in leadership positions often seen as charismatic, he regularly feels the pressure to succeed. A fear of failure is often is the catalyst that propels him to success.

“The Determinator” is capable of great leadership in spite of the self-doubt that is often successfully cloaked from others for fear of rejection. This is always a major concern of this Personality Type. He is a keen observer of the faults and frailty in others, which is often the primary difficulty in both personal and professional relationships. He is capable of intimacy however; it is usually transitory and elusive. It doesn't last for long, as he strives for perfection in himself and others. He is capable of giving up that what he has for that which he thinks he wants and might even find.

He is often admired and infrequently understood, he is reliable, dynamic and a force to be reckoned with in society.

Statement Of The NVL On The Symbolic Level For Personality Type 3.12 “I feel the pain of aloneness, I turn those feelings of sadness inward and Intellectualize and deny them.”

Examples of the specific non-verbal responses, or NVLs, that are observable and that can be used to lead to a unique diagnosis or classification into one of the twelve personality types include:

### NVL Type One (1-1)

1) Eyes Open
2) Eyes Not Focused (Staring/Not Present)
3) Slack Jaw (Mouth Not Closed)
4) Tongue Showing (Big Tongue, In The Mouth)
5) Lowering Of Head
6) Shallow Breathing
7) Shoulders Downward
8) Eyes Teary
9) Corners Of Mouth Downward
10) Swallow Down
11) Eyes Closing
12) Eyes Open
SL-1 Pain
SL-2 Sadness in or Anger in (Key is inward)
SL-3 Denial by swallowing down and trance

### NVL Type Two (2-2)

1) Eyes Open
2) Eyes Wide Open
3) Pronounced Jaws Sometimes Pulsing
4) Tongue Out Quick
5) Tightening Of The Mouth
6) High Breathing
7) Lines Throat Deeper
8) Concentration Lines Stronger
9) Movement Of The Shoulders
10) Eyes Close Not Completely (More One Eye Not Completely Closed)
11) Eyes Open
SL-1 Shock/FEAR
SL-2 Anger Out
SL-3 ACTING OUT, DISTRUST, CONTROL BY CLENCHING JAWS

### NVL Type Three (3-3)

1) Eyes Open
2) Eyebrows Up
3) Eyes Closed
4) Eyes Open
5) Teariness And Tightening Eyelids (Squinting)
6) Somewhat Developed Jaws
7) Tension Upper Lip
8) Pressing Lips
9) Blocked Breathing
10) Tongue Out/In
11) Eyes Open
SL-1 Disbelief/Pain
SL-2 Anger
SL-3 CONTROL CER

### NVL Type Four (1.4)

1. Eyes open
2. Eyes closing, not complete
3. Teary eyes
4. Eyes not focused
5. Mouth not fully closed
6. Lines from the corner of the mouth downwards
7. Tension around the mouth
8. Eyebrow(s) up
9. Block in throat
10. Eyes open (distanced)
SL-1 Denial
SL-2 Sadness (seen by teary eyes)/Anger (seen by cynicism)
SL-3 Control by holding back and trance

### NVL Type 5 (1.5)

1. Eyes open
2. Teary partially open eyes
3. Eyes close
4. Biting down
5. Lines bilateral nose and mouth intensified
6. Tension round the mouth
7. Swallow down
8. Eyes open
SL-1 Pain
SL-2 Anger
SL-3 Control, swallowing down

### NVL Type Six (2.6)

1. Eyes open
2. One Eye Shock
3. One Eye shows tears
4. Concentration lines
5. Eyes close
6. Developed masseter
7. Tension around mouth
8. Jaw shifts
9. Swallowing down
10. Eyes open
SL-1 Shock/Pain
SL-2 Anger/sadness
SL-3 Control by Distancing and Swallowing Down

### NVL Type Seven (2.7)

1. Eyes open
2. Eyes wide open
3. Eyes close (may not completely close)
4. Masseter muscle pulsing and/or tongue out
5. Tighten top lip
6. Swallow down
7. Eyes open
SL-1 Shock
SL-2 Anger-Out
SL-3 control by biting down, CER

### NVL Type Eight (3.8)

1. Eyes open
2. Teary eyes (squinting)
3 One eye more open than the other
4 Jaw shifting
5. Tighten top lip
6. Biting down
7. Swallow down
8. Eyes open
SL-1 Pain
SL-2 Anger In
SL-3 Distancing, Biting down

### NVL Type Nine (3.9)

1. Eyes open
2. Eyes shiny
3. Shifting of jaws
4. Eyebrow(s) up
5. Building up pressure around mouth
6. Mouth open
7. Showing teeth
8. Mouth closed
9. Eyes closing, but not completely (distrust)
10. Swallow down
11. Eyes open
SL-1 Pain/Fear
SL-2 Anger Out
SL-3 Cynicism, Swallowing Down, CER

### NVL Type Ten (1.10)

1. Eyes open
2. Eyes not focused
3. Eyebrow (s) up
4. Masseter muscle slightly developed
5. Shallow breathing
6. Lines from the corner of the mouth downwards
7. Pouting lower lip
8 Corners of mouth up (sardonic feeling)
9. Swallowing Down
10. Eyes open
SL-1 Pain
SL-2 Anger In/Out
SL-3 Trance, Seduction using CER

### NVL Type 11 (2.11)

1. Eyes open
2. Eyes shiny and wide open
3. Shifting of jaws
4. Pressure Built up around the mouth
5. Mouth open
6. Teeth Showing
7. Mouth closed
8. Eyes closing, but not completely (distrust)
9. Swallow down
10. Eyes open
SL1: Shock
SL2: Anger out
SL3: Seduction by CER and control by biting down

### NVL Type Twelve (3.12)

1. Eyes open
2. Eyebrows lifted
3. Teariness
4. Eyes larger
5. Eyes closed
6. Eyes open
7. Developed jaws a-symmetric
8 Biting on self
9. Pressure on lips
10. Swallowing down
11. Eyes open
SL-1 Disbelief/Denial
SL-2 Anger In and Sadness In
SL-3 Intellectualized aggression control by swallowing down

### Example—Saddam Hussein's Non-Verbal Leak

A close look at former Iraqi dictator Saddam Hussein's NVL can give a good example of analyzing the NVL to the symbolic levels and then through to the resultant personality type. Saddam exhibited behaviors of a cruel, intimidating, murderous psychopath, but the present method reveals some things that are not common knowledge. This specific example illustrates this method's effectiveness as a tool for forensic examiners.

Based on Saddam's known actions and his NVL, along with the diagnostic criteria, it is clear that he fit the clinical category of a psychopath. Saddam adhered to the pattern or process impulse—thought—action, whereas other people use impulse—thought-strategy—action. Thus, he would have known what one thought but not cared what one felt. It is clear that he was a psychopath with paranoid features. Although there was a real threat, his behavior could plainly be classified as paranoid. While his notorious behavior shows that he was psychopathic, his non-verbal behavior was also a clue leading to this deduction.

### Saddam's Non-Verbal Leak

Saddam elevated his left eyebrow pulling his eyes into a tight stare, then tightened his jaw muscles and proceeded to tighten his upper lip and swallow down. This NVL would be listed as follows:

Eyes open

Left Eyebrow elevated

Eyes into a tight stare

Tightened jaw muscles

Tightened upper lip

Swallowed down

Eyes open

### Saddam's Non-Verbal Leak Brought to a Symbolic Level

The referential aspect of Hussien's non-verbal leak is a tool that provides clear distinctions between body functions and the conscious and unconscious self-concepts. The fact that the unconscious can be seen via the NVL in the face and body eliminates guessing about the movement, meaning, and messages of Saddam or virtually anyone else. The muscles of his face provide clues to the true message of his unconscious. Muscles controlling the expressions around his mouth reflect, as part of the SL-3 in the NVL, anger in. The muscles affecting the closing and pursing of his lips are associated with his SL-3 control as it relates to his unconscious self-concept.

The principal muscle associated with the control and blocking of his aggression is the masseter. As his primary SL-3 was the blocking of his aggressive impulses, he developed enlarged masseter muscles. These muscles were enlarged due to his literal biting down of his initial response (SL-2) and diverting it to his SL-3 for later expression in a calculated emotional response. There was tension showing in his top lip as he literally kept a stiff upper lip, keeping his mouth shut about what he really felt.

Saddam's NVL had aspects of what we refer to as no behavior (point 3 of his NVL, “Eyes into a tight stare”). This makes analysis of non-verbal behavior difficult in cases such as Saddam. It is a characteristic of a psychopath to exhibit little, if any, noticeable response in situations where other people would show their feelings clearly. The no-behavior behavior results from the lack of super ego development, which is a hallmark of the psychopath. Conversely, it can also be found in individuals with a weak ego structure. In the case of Saddam, it was the ticking time bomb inside, waiting to explode, which, as history shows, detonated all too often.

Without the super ego, the person lacks what could be termed the conscience, the internal check that keeps the cultured gentleman from becoming the unspeakable fiend.

### Saddam's Symbolic Level of Responses:

SL-1 Impact—pain/disbelief
SL-2 Primary Emotion—anger
SL-3 Primary Coping Strategy—calculated emotional response

### The NVL Statement on the Symbolic Level

We can deduce that Saddam might say, “I feel distrust and disbelief, and it makes me angry. I, however, control my anger and search for a rationalization to give it open and full expression.” How is this deduced from his NVL? We can deduce “I feel distrust and disbelief” from Saddam's point 2, left eyebrow elevated, and 3, eyes in a tight stare. We garner the statement “It makes me angry” from point 4, the tightening of his jaw muscles. Point 5, the tightening of his upper lip, reveals “I, however, control my anger,” and point 6, swallowing down, provides us with “I search for a rationalization to give it open and full expression.” Saddam's clinical diagnosis, like all evaluations done using this method, is based solely on his nonverbal responses and the symbolic levels of his NVL.

Saddam's SL-1, -2, and -3 results do not suggest that a person with similar responses to the world will adopt the same strategies as the Butcher of Baghdad. They reveal that when extreme behavior is known to exist and a criminal profiler is called into a case, he or she is able to deduce the unconscious strategies and predict the behavior and actions of the person being profiled with a higher degree of exactness. The profiler strives for accuracy and predictability, so he or she can provide practical suggestions for law enforcement.

The NVL is based on observable data. This, of course, is not possible when there is no known photograph or video record of the subject available. In most cases, some description eventually surfaces during the investigation.

### Saddam's Clinical Profile

The psychopath does not develop a capacity for depressive anxieties or for guilt from concern that his actions can hurt others. This explains the lack of response on a nonverbal level. Saddam's swing from the rifle-firing leader on a balcony to the benevolent man stroking a frightened child's head on CBS television demonstrates the range of possibilities that typify the psychopath. He demonstrated his unique management style in a meeting when a general gave unwelcome advice. Saddam ordered him to stand and then shot him six times. Stories abound regarding Saddam's violent actions and his orders for others to commit violent actions. Consider the actions for which he was tried and hanged:

Saddam's NVL showed he had an unconscious core of distrust and disbelief. The consequence was that no one could get close to him. It was also a factor in the development of antisocial strategies that manifested themselves so clearly in cold, calculated, vicious behavior. For example, Saddam unemotionally and with absolute assurance, systematically manipulated his son-in-laws to return from Jordan, where they had fled. They feared retribution after making remarks about his weapons program.

In the present method of analysis there are 12 hypnotized personality types. In the methodology, Saddam is within personality type seven. It is important to note that not all people who are within this category are ruthless tyrants or, for that matter, psychopaths. Saddam's case is that of a personality at the extreme limits of psychopathology so that his actions resulted in behavior with a proclivity to a specific clinical diagnostic category. In his case, it is an extreme manifestation of a type seven.

The type seven, or observer personality, is a witness not taking part in the events that go on around him or her. This ability to stand back and observe individuals and events makes him or her a powerful person when it comes to facts, information, and data about people and places. He or she is often a critical player in major decision-making situations and is often found at the center of policy decisions. His or her strong ego often puts him or her in the center of the fray where action is required. A type seven personality knows what is necessary to obtain his or her objectives, but often watches others' actions rather than leading a group. He or she is a keen observer and is insightful about the dynamics of the situations in which he or she becomes involved. He or she will not hesitate to speak up when he or she feels the time is right, and he or she quickly notices who agrees with his or her viewpoint and who does not. When such persons want to be, they can be the center of attention, showing power, skills, and outstanding organization abilities.

What is claimed is:

1. A method of diagnosing a psychological condition in an individual comprising the steps of:
placing the individual in direct view of a display device having a real-time frontal image of the individual and/or a recording device for review at a later time;
asking the individual a plurality of questions designed to bring out an emotional response in the individual while the individual watches the real-time image generated on the display and/or while recording the individuals image;
observing all repetitive physical movements by the individual while the individual is responding to the questions asked;
identifying from the observed repetitive physical movements nonverbal leaks (NVL) reflecting an unresolved perceived trauma in the individual; and
diagnosing a psychological condition by correlating the individual's NVL to a psychological condition or an underlying perceived trauma.

2. The method of claim 1, further comprising the step of hypnotizing the individual and asking the individual further questions while hypnotized after identifying the individual's NVL.

3. The method of claim 1, further comprising decoding the unconscious meaning of the NVL using one or more symbolic levels, including: Symbolic Level One, (SL-1), the impact of a perceived traumatic event on the individual; Symbolic Level Two (SL-2), the primary emotion the individual has to the perceived traumatic event; and Symbolic Level Three (SL-3), the primary coping strategy to the perceived traumatic event.

4. The method of claim 3, wherein the SL-1 impact is manifested by way one of the following: shock (eyes large), fear (teariness), denial (eyes up, trance, white below eyes), disbelief (eyes closing), pain (tearing, turning away from) and trance (eyes that stare in an unfocused way); the SL-2 primary emotion.

5. The method of claim 3, further comprising identifying the perceived traumatic event.

6. The method of claim 1, wherein the display device is a television or computer monitor and asking the questions at least three times in varied context.

7. The method of claim 1, wherein the real-time frontal image of the individual is generated by a video recorder or digital camera recording the image of the individual.

8. The method of claim 1, wherein a recording is made of the individual during questioning and the step of identifying the individuals NVL comprises sitting with the individual and watching the recording using slow motion and/or still frame to correlate the repetitive physical movements with the questions asked.

9. The method of claim 8, wherein the recording of the image of the individual is from the shoulders up.

10. The method of claim 9, wherein the NVL comprises at least one repetitive physical movement that occurs during questioning selected from the group consisting of: eyes held open, eyes not focused; slack jaw, tongue showing, lowering of head, shallow breathing, shoulders downward, eyes teary, corners of mouth downward, eyes closing, eyebrows up, eyes closed, teary eyes, squinting, tightening of jaw muscles, tightening of upper lip, pressing lips, holding breath, moving tongue in and out, holding mouth open, tightening around the mouth, moving eyebrow(s) up or down, biting down, swallowing, shifting jaw, showing teeth, eyes closing partially, and biting lip or tongue.

11. The method claim 10, wherein the individual is asked to at least three times repeat their name and the name of their mother, father, or someone important to them.

12. The method of claim 1, wherein the psychological condition is: a mood disorder, an anxiety disorder, a somatoform disorder, a factitious disorder, a sleep disorder, an eating disorder, an impulse/control disorder, an adjustment disorder, or a personality disorder.

13. The method of claim 1, further comprising treating the individual after diagnosing the psychological condition.

14. The method of claim 13, wherein the treating of the individual comprises identifying the underlying unresolved perceived trauma in the individual and assisting the individual to resolve the perceived trauma.

15. The method of claim 1, further comprising a screening session prior to placing the individual in direct view of a display device.

16. The method of claim 15, wherein the screening session comprises a face to face meeting between the individual and a therapist.

17. A method of diagnosing the underlying cause of an unresolved perceived trauma in an individual condition comprising the steps of:
placing the individual in direct view of a display device having a real-time frontal image of the individual;
asking the individual a plurality of questions designed to bring out an emotional response in the individual while the individual watches the real-time image generated on the display;
observing all repetitive physical movements by the individual while the individual is responding to the questions asked;
identifying from the observed repetitive physical movements nonverbal leaks (NVL) reflecting an unresolved perceived trauma in the individual; and
diagnosing a psychological condition by correlating the individual's NVL to a psychological condition or an underlying perceived trauma.

18. A method of assigning a personality type to an individual comprising the steps of:
asking the individual a plurality of questions designed to bring out an emotional response in the individual;
observing all repetitive physical movements by the individual while the individual is responding to the questions asked;
identifying from the observed repetitive physical movements nonverbal leaks (NVL) reflecting an unresolved perceived trauma in the individual; and
assigning a personality type to the individual by correlating the individual's NVL to a personality type.

19. The method of claim 18, further comprising the step of assigning the individual one of three basic personality distinctions selected from a pre-matrix, a during matrix, and a post matrix based on the identified NVL the individual expressed.

20. The method of claim 19, further comprising the step of assigning the individual one of twelve sub personality type distinctions as follows: for individuals having a personality type assigned to the pre-matrix group a sub personality type selected from the group: the planner, the plotter, the detailer, and the influencer; or for individuals having a personality type assigned to the during matrix group a sub personality type selected from the group: the doer, the thinker, the pouncer, and the prevailer; or for individuals having a personality type assigned to the post matrix group a sub personality type selected from the group: the doubter, the analyst, the inquisitor, and the determinator.
